(19) United States
(12) Patent Application Publication
(10) Pub. No.: US 2011/0092829 A1
(43) Pub. Date: Apr. 21, 2011

(54) BLOOD PRESSURE MONITOR AND METHOD FOR MEASUREMENT OF BLOOD VESSEL HARDENING

(75) Inventors: WEI-CHIH HU, CHUNG LI (TW); LIANG-YU SHYU, CHUNG LI (TW); YUAN-TA SHIH, CHUNG LI (TW); YI-JUNG SUN, CHUNG LI (TW); CHEN-HUAN CHEN, TAIPEI (TW); HAO-MIN CHENG, TAIPEI CITY (TW)

(73) Assignee: CHUNG YUAN CHRISTIAN UNIVERSITY, CHUNG LI (TW)

(21) Appl. No.: 12/716,572

(22) Filed: Mar. 3, 2010

(30) Foreign Application Priority Data
Oct. 15, 2009 (TW) 098134870

(51) Int. Cl. A61B 5/022

(52) U.S. Cl. 600/490

(57) ABSTRACT

A blood pressure monitor and a method for detecting vascular sclerosis thereof are revealed. The blood pressure monitor includes a cuff, an air pump, an air escape valve, a pressure sensor, a processing circuit, and an arithmetic circuit. The cuff is arranged at a body to be detected and the air pump inflates the cuff. The air escape valve is for releasing air from the cuff while the pressure sensor arranged at the cuff measures a cuff pressure to generate an analog pressure sensing signal. The processing circuit processes the pressure sensing analog signal and generates a digital pressure sensing signal. Then a systolic pressure and a diastolic pressure of the detected body are calculated according to the digital pressure sensing signal. The degree of blood vessel hardening is checked according to a systolic area of the calculated systolic pressure and a diastolic area of the calculated diastolic pressure.

### BACKGROUND OF THE INVENTION

1. Field of Invention

The present invention relates to a measurement method, especially to a blood pressure monitor and a method for measurement of blood vessel hardening.

2. Description of Related Art

Due to lives under high pressure and delicate foods, high blood pressure has become one of the ten leading causes of death. People not only have to monitor their blood pressure but also control the food intake for prevention of high blood pressure. In recent years, cardiovascular disease has also been one of the ten leading causes of death and has being with an increasing rate according to statistics of the department of health. The cardiovascular disease refers to arterial disease (atherosclerosis) so that a hardening of blood vessels (vascular sclerosis) is one of important indicators of cardiovascular diseases. Once the hardening of blood vessels is discovered early, the cardiovascular disease can be prevented. Thus people got to monitor their blood pressure and the degree of blood vessel hardening so as to check their health conditions. Therefore, both high blood pressure and cardiovascular diseases can be prevented.

Along with increasing incomes, change of population structure, adoption of new medical technology, and some other factors, people have paid more attentions to health and medical and health devices such as blood pressure monitors, glucosemeters, etc., have been essentials for families. Thus it is convenient for users to measure their blood pressure and blood glucose so as to learn their health conditions for disease prevention. Although the medical technology is quite advanced now, there is still no easy way to measure the degree of blood vessel hardening, or an index of vascular stiffness. Thus there is no good measure of vascular stiffness assessment of health conditions. Therefore, cardiovascular disease remains one of the ten leading causes of death. Blood pressure (BP) and blood pressure waveforms are used as indicators for evaluating cardiac functions yet a plurality of physiological mechanisms has effects on blood pressure and its waveform. A common blood pressure monitor used now includes a cuff that measures the pressure of blood vessels. The cuff is inflated to a preset pressure by an electric pump and then the electric pump is controlled by a microprocessor so as to make the amount of air released from the cuff equal to the amount of air inflated into the cuff. Thus the pressure inside the cuff remains in a low pressure state for continuously measuring blood pressure signals.

A conventional way of diagnosis is an intrusion-detection way. The procedures are not only complicated but also time-consuming. Thus the most common index of arterial stiffness adopted now is Pulse Wave Velocity (PWV). The systolic pressure and the diastolic pressure of arteries now are determined by an oscillometric method described in the articles. However, the method provides no guarantee of accuracy in all conditions because it is based on clinical statistics. Once the measured patients with cardiovascular diseases, the systolic pressure and the diastolic pressure may be overestimated or underestimated. The blood pressure monitors available on the market determines an average blood pressure according to a pressure value of a point on the oscillating waveform that reaches a maximum amplitude. And the systolic pressure is defined as a pressure of a point on the waveform reaching about 50% maximum amplitude appeared before the waveform arrives the maximum amplitude while the diastolic pressure is defined by a point having about 50% maximum amplitude on the waveform after the waveform arrives the maximum amplitude. This is the oscillometric method now used for automatic blood pressure measurement. The method is to measure mean blood pressure of the patients and is unable to provide doctors with accurate data for diagnosis.

Thus there is a need to provide a blood pressure monitor and a method for measurement of vascular sclerosis that overcomes above shortcomings.

### SUMMARY OF THE INVENTION

Therefore it is a primary object of the present invention to provide a blood pressure monitor and a method for measurement of vascular sclerosis that calculates a systolic pressure and a diastolic pressure of a person measured and further checks the degree of vascular sclerosis by a systolic area associated with a systolic pressure, and a diastolic area associated with a diastolic pressure.

In order to achieve above objects, a method for measurement of vascular sclerosis according to the present invention includes following steps. At first, set a cuff on a body to be detected. Inflate the cuff by an electric air pump to make the cuff expand and then deflate the cuff. While deflating the cuff, measure a pressure of the cuff and generate an analog pressure sensing signal. Next process the analog pressure sensing signal to generate a digital pressure sensing signal and convert the digital pressure sensing signal. Calculate a systolic pressure and a diastolic pressure of the detected body according to the converted digital pressure sensing signal. At last, measure the vascular sclerosis according to area size of a systolic area associated with a systolic pressure and area size of a diastolic area associated with a diastolic pressure of the detected body.

Moreover, a blood pressure monitor of the present invention further includes an instrumentation amplifier and a filter. The instrumentation amplifier amplifies the pressure sensing signal generated by the pressure sensor while the filter is coupled with the instrumentation amplifier for filtering the pressure sensing signal amplified by the instrumentation amplifier. Then the processed pressure sensing signal is sent to the first conversion circuit for conversion.

Furthermore, a blood pressure monitor of the present invention further includes a second conversion circuit that is coupled with the arithmetic circuit and is able to receive, convert both an inflation control signal and a deflation control signal from the arithmetic circuit, and send the signals to the air pump and the air escape valve respectively for control of the air pump and the air escape valve to inflate and deflate the cuff.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of an embodiment of a blood pressure monitor according to the present invention;

FIG. 2 is a flow chart of an embodiment of a method for measurement of blood vessel, hardening according to the present invention;

FIG. 3 is a block diagram of another embodiment of a blood pressure monitor according to the present invention;

FIG. 4 is a flow chart of another embodiment of a method for measurement of blood vessel hardening according to the present invention.

### DETAILED DESCRIPTION OF THE PREFERRED EMBODIMENT

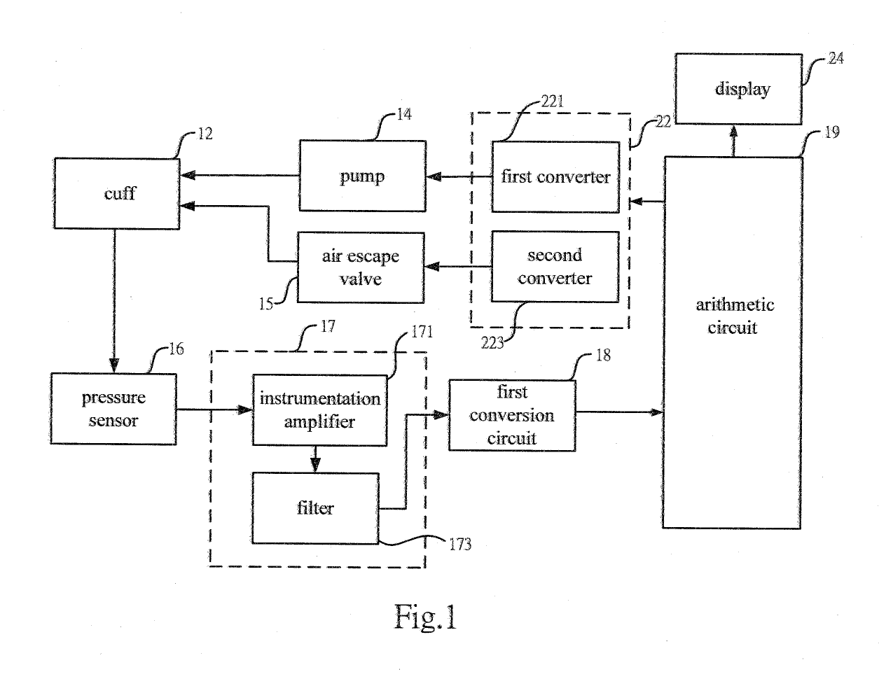

Refer to FIG. 1, a blood pressure monitor according to the present invention includes a cuff 12, an air pump 14, an air escape valve 15, a pressure sensor 16, a processing circuit 17, a first conversion circuit 18, an arithmetic circuit 19, a second conversion circuit 22 and a display 24. The cuff 12 is arranged at people's hands and is pumped up and inflated by the air pump 14 connected therewith. In this embodiment, the air pump 14 is an electric air pump that inflates the cuff 12 in a linear way. The air escape valve 15 is coupled with the air pump 14 so as to release air in the cuff 12. In this embodiment, the air escape valve 15 is an electric valve or a linear valve that releases air from the cuff 12 in a linear way. In this embodiment, the body detected is people's hand but not limited to human bodies. The body to be detected can also be an animal body.

As shown in FIG. 1, the pressure sensor 16 is disposed on the cuff 12 for measuring pressure of the cuff 12 and generating an analog pressure sensing signal that is a waveform signal. The processing circuit 17 is coupled with the pressure sensor 16 and is for processing the analog pressure sensing signal to generate a digital processed signal which is also a waveform signal. The processing circuit 17 mainly deals with analog pressure sensing signals such as amplifying the waveform signals and filtering noises of the waveform signals for convenience of following processes such as conversion and calculation of the first conversion circuit 18 and the arithmetic circuit 19 so as to increase the accuracy. In an embodiment of the present invention, the processing circuit 17 is an analog processing circuit.

In this embodiment, the processing circuit 17 includes an instrumentation amplifier 171 and a filter 173. The instrumentation amplifier 171 is coupled with the pressure sensor 16 to amplify the analog pressure sensing signal while the filter 173 coupled with the instrumentation amplifier 171 is for filtering the amplified analog pressure sensing signal. If the noise-to-signal ratio is not high, the analog pressure sensing signal generated from the pressure sensor 16 is amplified by the instrumentation amplifier 171 and then is directly sent to the first conversion circuit 18, without disposition of the filter 173. The above embodiment is only a preferred embodiment of the present invention. The design of the instrumentation amplifier 171 varies according to different kinds of pressure sensors 16, the state of the analog pressure sensing signal or requirements of the arithmetic circuit 19.

Still refer to FIG. 1, the first conversion circuit 18 for conversion of pressure sensing signals from analog pressure sensing signals to digital pressure sensing signals is coupled with the processing circuit 17. In an embodiment of the present invention, the first conversion circuit 18 is an analog-to-digital converter that samples waveform of the processed signal and outputs the sampled result which is a digital signal. The arithmetic circuit 19 coupled with the first conversion circuit 18 is used to receive the processed signal being converted by the first conversion circuit 18 and then calculate a systolic area associated with a systolic pressure, and a diastolic area associated with a diastolic pressure of the human body according to pressure changes of the cuff 12. And the degree of blood vessel hardening is checked according to area size of the systolic area of the systolic pressure and of the diastolic area of the diastolic pressure.

Moreover, the arithmetic circuit 19 is coupled with the display 24 so as to send the measured data of the systolic pressure and the diastolic pressure to the display 24 for users to read. Furthermore, according to the received processed signal, the arithmetic circuit 19 obtains and sends a pulse rate to the display 24 for display. In this embodiment, the display 24 is a liquid crystal display (LCD).

In addition, the arithmetic circuit 19 generates an inflation control signal and a deflation control signal for control of the air pump 14 and the air escape valve 15 respectively. The arithmetic circuit 19 in this embodiment is a microprocessor. Once the air pump 14 and the air escape valve 15 can only receive analog signals, the second conversion circuit 22 of the present invention can convert both the inflation control signal and the deflation control signal generated from the arithmetic circuit 19 into analog signals, respectively sent to the air pump 14 and the air escape valve 15. Thus the air pump 14 is controlled to inflate the cuff 12 and the air escape valve 15 is controlled to release air from the cuff 12.

The second conversion circuit 22 includes a first converter 221 and a second converter 223. In a preferred embodiment, the first converter 221 as well as the second converter 223 is a digital to analog converter. The first converter 221 is coupled between the arithmetic circuit 19 and the air pump 14 and is used for converting the inflation control signal generated by the arithmetic circuit 19 into an analog signal and sending the analog signal to the air pump 14 so as to control the air pump 14 for inflation of the cuff 12. The second converter 223 coupled between the arithmetic circuit 19 and the air escape valve 15 is for converting the deflation control signal generated by the arithmetic circuit 19 into an analog signal and sending the analog signal to the air escape valve 15 so as to control the air escape valve 15 for air releasing of the cuff 12.

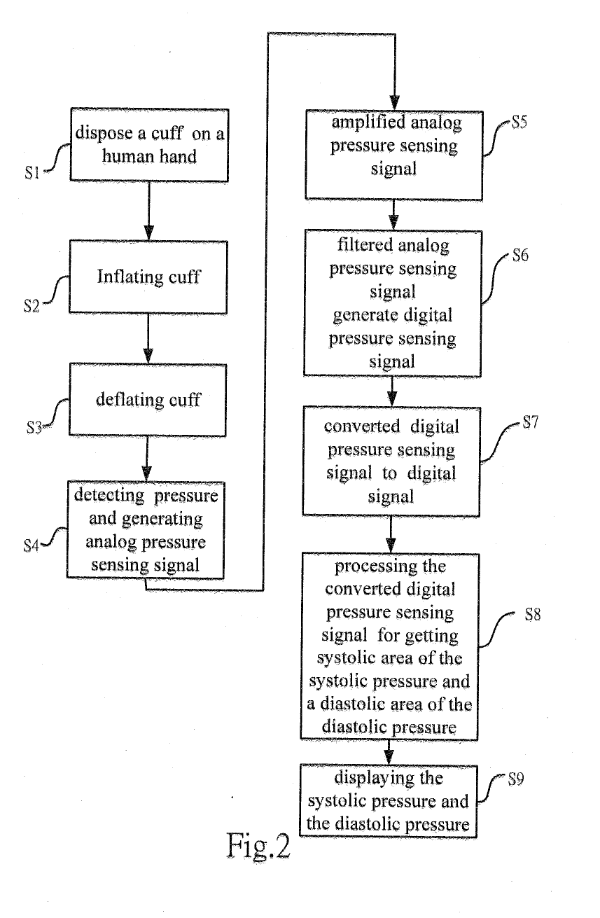

Refer to FIG. 2, a flow chart of a method for measurement of vascular sclerosis according to the present invention is revealed. As shown in figure, firstly take the step S1, dispose a cuff 12 on a human hand. Then as shown in the step S2, the cuff 12 is inflated by the air pump 14 that receives an inflation control signal generated from the arithmetic circuit 19. The arithmetic circuit 19 controls the air pump 14 to inflate in a linear way. Later, as shown in the step S3, the arithmetic circuit 19 generates and sends a deflation control signal to the air escape valve 15 so as to control the air escape valve 15 that releases air from the cuff 12. Thus the gas pressure inside the cuff 12 is decreasing gradually. The arithmetic circuit 19 controls the air escape valve 15 to deflate in a linear way. Next, refer to the step S4, the pressure sensor 16 detects a pressure of the cuff 12 and generates an analog pressure sensing signal correspondingly. The analog pressure sensing signal includes a plurality of waveform signals whose waveforms oscillate along with the pulse beat.

Next the analog pressure sensing signal is processed to generate a digital pressure sensing signal. As shown in the step S5 and the step S6, at first, the analog pressure sensing signal is amplified by the instrumentation amplifier 171 and then the amplified analog pressure sensing signal is filtered by the filter 173 so as to generate the digital pressure sensing signal. Then refer to the step S7, the digital pressure sensing signal is converted to a digital signal by the first conversion circuit 18. As shown in the step S8, the arithmetic circuit 19 processes the converted digital pressure sensing signal to get a systolic pressure and a diastolic pressure and measure the degree of blood vessel hardening according to a systolic area of the systolic pressure and a diastolic area of the diastolic pressure. For example, once the systolic area is larger than the diastolic area, the blood vessel hardening occurs. When the vascular sclerosis occurs, the blood flow is slower so that the area of that period is increased. Thus the degree of blood vessel hardening is checked according to the systolic area and the diastolic area. Moreover, as shown in the step S9, the systolic pressure and the diastolic pressure are displayed.

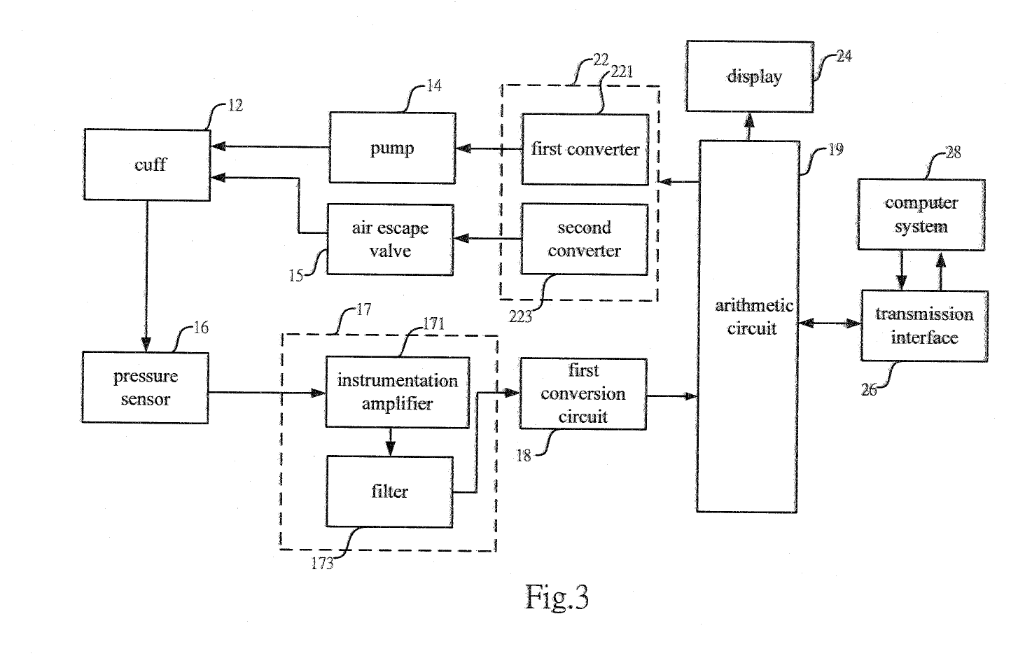

Refer to FIG. 3, a block diagram of another embodiment of a blood pressure monitor related to the present invention is revealed. The difference between this embodiment and the above one is in that this embodiment further includes a transmission interface 26 and a computer system 28. The transmission interface 26 is coupled with the arithmetic circuit 19 for sending the digital pressure sensing signal converted by the first conversion circuit 18 while the computer system 28 is coupled with the transmission interface 26 for receiving the digital pressure sensing signal from the arithmetic circuit 19 and then further processing and analyzing the digital pressure sensing signal. For example, the waveform of the pressure sensing signal generated from the pressure sensor 16 is shown on a display of the computer system 28 or further analysis of the waveform is carried out for other measurement requirements. In a preferred embodiment of the present invention, the transmission interface 26 is a Universal Serial Bus (USB) or other interface with general specifications.

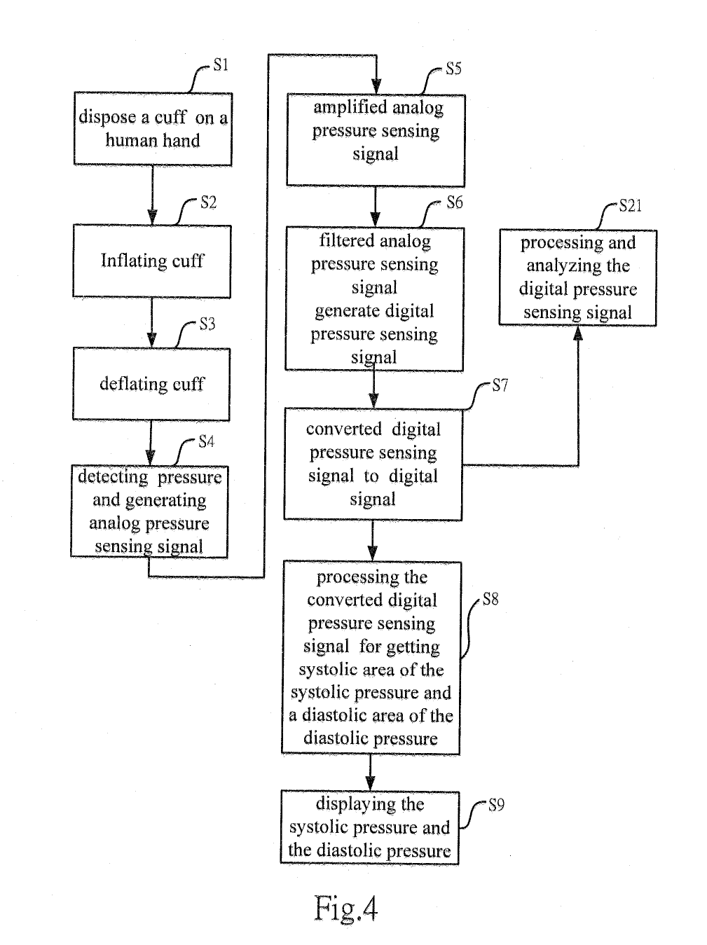

Refer to FIG. 4, another embodiment of the present invention is disclosed. As shown in figure, the difference between this embodiment and the above one is in that this embodiment further includes a step S21, the processed digital pressure sensing signals are sent to the computer system 28 through the transmission interface 26. The computer system 28 receives the digital pressure sensing signals and further processes and analyzes the digital pressure sensing signals.

In summary, a blood pressure monitor and a method for measurement of blood vessel hardening thereof includes the following steps. A cuff is disposed on a body to be detected. The cuff is connected with an air pump to be inflated while an air escape valve is coupled with the air pump for releasing air from the cuff. A pressure sensor is arranged at the cuff and is used for sensing cuff pressure so as to generate pressure sensing signals. A processing circuit processes analog pressure sensing signals generated by the pressure sensor to generate digital pressure sensing signals. An arithmetic circuit calculates a systolic pressure and a diastolic pressure of the detected body according to the digital pressure sensing signals. And the blood vessel hardening is further checked by a systolic area of the calculated systolic pressure and a diastolic area of the calculated diastolic pressure. Thus the blood pressure of the detected body is obtained. Compared with prior data of blood pressures not based on physical laws, data got by the present invention is with higher accuracy.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A method for measurement of blood vessel hardening comprising the steps of:
arranging a cuff at a body to be detected,
inflating the cuff,
deflating the cuff and simultaneously sensing pressure of the cuff to generate a plurality of analog pressure sensing signals,
processing the analog pressure sensing signals to generate a plurality of digital pressure sensing signals and then converting the digital pressure sensing signals to a plurality of blood pressure values, and
calculating the digital pressure sensing signals to get a systolic pressure and a diastolic pressure and checking the degree of vascular sclerosis by a systolic area of the calculated systolic pressure, and a diastolic area of the calculated diastolic pressure.

2. The method as claimed in claim 1, wherein the step of processing the analog pressure sensing signals to generate a plurality of digital pressure sensing signals further includes the steps of:
amplifying the analog pressure sensing signals, and
filtering the amplified analog pressure sensing signals to generate the digital pressure sensing signals.

3. The method as claimed in claim 2, wherein the step of filtering the amplified analog pressure sensing signals to generate the digital pressure sensing signals further includes a step of:
converting the digital pressure sensing signals and processing the converted digital pressure sensing signals.

4. The method as claimed in claim 1, wherein the step of calculating the digital pressure sensing signals to get a systolic pressure and a diastolic pressure and checking the degree of vascular sclerosis by a systolic area of the calculated systolic pressure, and a diastolic area of the calculated diastolic pressure further includes a step of:
calculating a pulse rate of the body to be detected according to the digital pressure sensing signal.

5. The method as claimed in claim 4, wherein the method further includes a step of: displaying the average blood pressure and the pulse rate.

6. The method as claimed in claim 1, wherein the method further includes steps of:
transmitting the digital pressure sensing signals to a computer system, and
processing and analyzing the digital pressure sensing signals.

7. The method as claimed in claim 1, wherein in the step of inflating the cuff, the cuff is inflated in a linear way.

8. The method as claimed in claim 1, wherein in the step of deflating the cuff, the cuff is deflated in a linear way.

9. A blood pressure monitor comprising:
a cuff disposed on a body to be detected,
an air pump connected with the cuff and used for inflation of the cuff,
an air escape valve coupled with the air pump and used for releasing air from the cuff,
a pressure sensor arranged at the cuff to detect pressure of the cuff while releasing air from the cuff for generating a plurality of analog pressure sensing signals,
a processing circuit coupled with the pressure sensor, processing the analog pressure sensing signals and generating a plurality of digital pressure sensing signals that are converted to a plurality of blood pressure values, and
an arithmetic circuit that calculates a systolic pressure and a diastolic pressure according to the digital pressure sensing signals and checking the degree of vascular sclerosis by a systolic area of the calculated systolic pressure, and a diastolic area of the calculated diastolic pressure so as to get an average blood pressure, the systolic pressure and the diastolic pressure of the body detected.

10. The device as claimed in claim 9, wherein the blood pressure monitor further includes:
a first conversion circuit coupled with the processing circuit and converting the digital pressure sensing signals.

11. The device as claimed in claim 9, wherein the processing circuit includes:
an instrumentation amplifier that amplifies the analog pressure sensing signals, and
a filter coupled with the instrumentation amplifier and filtering the analog pressure sensing signals amplified by the instrumentation amplifier so as to generate the digital pressure sensing signals.

12. The device as claimed in claim 9, wherein the processing circuit is an analog processing circuit.

13. The device as claimed in claim 9, wherein the air escape valve is an electric air escape valve.

14. The device as claimed in claim 9, wherein the air escape valve is a linear air escape valve.

15. The device as claimed in claim 9, wherein the air pump is an electric air pump.

16. The device as claimed in claim 10, wherein the first conversion circuit is an analog-to-digital converter that converts the analog pressure sensing signals to digital pressure sensing signals.

17. The device as claimed in claim 9, wherein the arithmetic circuit is a microprocessor.

18. The device as claimed in claim 9, wherein the arithmetic circuit calculates a pulse rate of the body to be detected according to the converted digital pressure sensing signals.

19. The device as claimed in claim 9, wherein the blood pressure monitor further includes:
a transmission interface coupled with the arithmetic circuit for sending the digital pressure sensing signals; and
a computer system coupled with the transmission interface and receiving the digital pressure sensing signals to process and analyze the digital pressure sensing signals.

20. The device as claimed in claim 19, wherein the transmission interface is a universal serial bus (USB).

21. The device as claimed in claim 9, wherein the blood pressure monitor further includes:
a display coupled with the arithmetic circuit and used for receiving and displaying the systolic pressure and the diastolic pressure.

22. The device as claimed in claim 21, wherein the display is a liquid crystal display (LCD).

23. The device as claimed in claim 9, wherein the blood pressure monitor further includes:
a second conversion circuit that is coupled with the arithmetic circuit for receiving an inflation control signal and a deflation control signal from the arithmetic circuit and then converts and sends the inflation control signal and the deflation control signal to the air pump and the air escape valve respectively for control of the air pump and the air escape valve.

24. The device as claimed in claim 23, wherein the second conversion circuit includes:
a first converter coupled between the arithmetic circuit and the air pump and used for converting and sending the inflation control signal from the arithmetic circuit to the air pump; and
a second converter coupled between the arithmetic circuit and the air escape valve and used for converting and sending the deflation control signal from the arithmetic circuit o the air escape valve.

25. The device as claimed in claim 24, wherein the first converter and the second converter are both digital to analog converters, respectively converting the inflation control signal and the deflation control signal to analog signals.
